(19) United States
(12) Patent Application Publication
(10) Pub. No.: US 2022/0195531 A1
(43) Pub. Date: Jun. 23, 2022

(54) EXOSOMES CONTAINING RNA WITH SPECIFIC MUTATION

(71) Applicant: APPLIED STEMCELL, INC., Milpitas, CA (US)

(72) Inventors: Vigneshwaran MANI, Mountain View, CA (US); Gianluca ROMA, Belmont, CA (US)

(21) Appl. No.: 17/441,692

(22) PCT Filed: Mar. 23, 2020
(86) PCT No.: PCT/US2020/024122

§ 371 Date: Sep. 21, 2021

Related U.S. Application Data
(60) Provisional application No. 62/822,037, filed on Mar. 21, 2019.

(51) Int. Cl. C12Q 1/6886; C12N 15/90; C12N 5/09; C12Q 1/6806

(52) U.S. Cl. CPC C12Q 1/6886; C12N 15/907; C12N 2510/02; C12Q 1/6806; C12Q 2600/156; C12N 5/0693

(57) ABSTRACT

Provided herein are methods for producing exosomes that contain RNA transcribed from a specific mutant gene or a transgene. In one embodiment, the method comprises the steps of: generating a cell comprising a mutation of a gene by using a site-specific nuclease; culturing the cell in a medium that allows the cell to secrete to the medium an exosome containing an RNA transcribed from the gene and comprising the mutation; and collecting the medium that contains the exosome. The exosomes generated can be used as reference material or therapeutic delivery device.

### CROSS-REFERENCE TO RELATED APPLICATIONS

This application claims priority to U.S. Provisional Application No. 62/822,037, filed Mar. 21, 2019, the disclosure of which is incorporated herein by reference.

### FIELD OF THE INVENTION

The present invention generally relates to cellular biology, diagnostics and therapeutics. More specifically, the present invention relates to methods for producing exosomes containing RNA with specific mutation and the uses of such exosomes.

### BACKGROUND

Exosomes, also known as extracellular vesicles, are cell-derived vesicles that are present in many eukaryotic fluids including blood, urine, cerebrospinal fluid, lavage and cultured medium of cell culture. Exosomes play a key role in processes such as coagulation, intercellular signaling, and waste management. There is a growing interest in the therapeutic and diagnostic applications of exosomes for oncology and other diseases. Exosomes are actively released from tumor cells that have shown to contain surface or molecular cargo biomarkers that include tumor-specific proteins, -small molecules, -nucleic acids (mRNA, microRNA, and DNA) that are indicative of the cancer progression and the stage. Specifically, exosome molecular cargo (proteins, nucleic acid, and small molecules) profiling have been subject of intense research for potential biomarkers for cancer. Currently, there exist only a few exosomal nucleic acid (exoRNA) biomarkers that have been realized for cancer diagnostics and treatment monitoring. This is due to the lack of exosome molecular reference standards for assay development, assay performance validation, and interpretation of results. Therefore, there is a need to develop exosome molecular references that mimic physical properties and genomic composition of exosomal cargo that are isolated from patient biofluids. This exosome molecular reference has a potential to be employed for assay development, limit-of-detection (LOD) assessment, quality assurance & proficiency testing to validate exosome-based clinical assay performance and understand cross-site and/or inter-operator reproducibility.

### SUMMARY OF THE INVENTION

In one aspect, the present disclosure provides a method for producing an exosome. In one embodiment, the method comprises the steps of: generating a cell comprising a mutation of a gene by using a genome editing enzyme; culturing the cell in a medium that allows the cell to secrete to the medium an exosome containing an RNA transcribed from the gene and comprising the mutation; and collecting the medium that contains the exosome.

In another embodiment, the method comprises the steps of: generating a cell comprising a transgene by using a genome editing enzyme; culturing the cell in a medium that allows the cell to secrete to the medium an exosome containing an RNA transcribed from the transgene; and collecting the medium that contains the exosome. In certain embodiments, the RNA is a microRNA, non-coding RNA, siRNA, mRNA, tRNA, rRNA, or shRNA.

In certain embodiment, the cell is generated from a cell line. In certain embodiments, the cell line is HCT116 or RKO. In certain embodiments, cell is generated from a stem cell. In certain embodiments, the stem cell is an induced pluripotent stem cell (iPSC).

In certain embodiments, the site-specific nuclease is a CRISPR/Cas nuclease, a zinc-finger nuclease (ZFN) or a transcription activator-like effector nuclease (TALEN).

In certain embodiments, the cell is homozygous in the mutation of the gene. In certain embodiments, the cell is heterozygous in the mutation of the gene. In certain embodiments, the gene is a cancer gene. In certain embodiments, the cancer gene is selected from the group consisting of EGFR, KRAS, BRAF, PIK3CA, AKT1, NRAS, HRAS, TP53, BRCA1, BRCA2, JAK2, RB1, PTEN, CTNNB1, APC, FLT3, KIT, ESR1, ERBB2, MAP2K1, FGR3, IDH1, IDH2, ATM, PIK3R1, FGFR2, PDGFRA, ABL1, FGFR1, GNA11, NOTCH1, GNAQ, GNAS, CDH1, CD2, MLH1, MET, ALK, RET, SMAD4, ROS1, BARD1, BRIP1, FBXW7, NBN, STK11, EML4-ALK, CD74-ROS1, KDR, APC, ALK, RAF1, MTOR, CHEK2, PLE, POLD1, KIF5B-ALK, CCDC6-RET, BCR-ABL1, and CD74-ROS1.

In certain embodiments, the mutation is a point mutation, an insertion, a deletion or a gene fusion. In certain embodiments, the mutation is selected from the group consisting of EGFR-T790M, EGFR-L858R, EGFR-V769_D770insASV, EGFR-E746_A750del, EGFR-E746_A750delELREA, EGFR-G719S, EGFR-L747_P753>S, EGFR-D761Y, EGFR-861Q, EGFR-S768I, EGFR-G719S, EGFR-C797S, KIT-D816V, PIK3CA-E45K, PIK3CA-H1047L, NRAS-Q61K, KRAS-G12D, BRAF-V600E, EML4-ALK (E13;A20, E6;A20, E20;A20), KIF5B-RET (K15;R12, K16;R12, K16;R12, K22;R12), CD74-ROS1 (C6;R34), EZR-ROS1 (E10;R34).

In certain embodiments, the method disclosed herein further comprises analyzing the exosome.

In certain embodiments, the method disclosed herein further comprises isolating the exosome from the medium. In certain embodiments, the method disclosed herein further comprises using the exosome as a reference, a quality control, or a proficiency panel.

In certain embodiments, the method disclosed herein further comprises isolating the RNA from the exosome. In certain embodiments, the method disclosed herein further comprises detecting the size of the RNA. In certain embodiments, the method disclosed herein further comprises using the RNA isolated from the exosome as a reference, a quality control, or a proficiency panel.

In certain embodiments, the method disclosed herein further comprises detecting a surface protein on the exosome. In certain embodiments, the surface protein is CD63.

In certain embodiments, the method disclosed herein further comprises detecting the mutation in the RNA. In certain embodiments, the mutation is detected using immuno-histochemistry (IHC), fluorescence in situ hybridization (FISH), PCR, Sanger sequencing or next generation sequencing. In certain embodiments, the mutation is detected using RT-PCR, digital PCR, or targeted next generation sequencing.

In certain embodiments, the method disclosed herein further comprises administering the exosome to a subject, e.g., as a therapeutic delivery device of RNA or protein.

In another aspect, the present disclosure provides an exosome produced according to the method disclosed herein.

In another aspect, the present disclosure provides a panel of exosomes, each produced according to the method as disclosed herein, wherein the panel of exosomes contains a panel of cancer specific RNA mutations in specified allelic frequency.

In yet another aspect, the present disclosure provides a kit comprising the exosome disclosed herein.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 illustrates a schema of the generation of exosome molecular reference material using CRISPR/Cas9 engineered cell lines.

FIG. 2A illustrates a workflow of the generation of exosome molecular reference.

FIG. 2B illustrates a workflow of the generation of engineered exosomes.

FIG. 3 illustrates an exemplary embodiment of dynamic light scattering size distribution analysis of exosomes isolated from engineered cells.

FIG. 4 illustrates an exemplary embodiment of size fragment analysis of ExoRNA derived from HCT116 cell line.

FIG. 5 illustrates an exemplary embodiment of the validation of cellular and exosome RNA mutant transcript levels.

FIG. 6 illustrates an exemplary embodiment of size profile of exosome RNA after lyophilization.

FIG. 7 illustrates an exemplary embodiment of ExoRNA size profile at day 0 and 6 months of storage for lyophilized exosomes.

FIG. 8 illustrates an exemplary embodiment of digital PCR confirmation of exoRNA EGFR transcript in lyophilized exosomes.

FIG. 9 illustrates an exemplary embodiment of digital PCR confirmation of exoRNA EGFR wildtype transcript in lyophilized exosomes after 6 months storage.

FIGS. 10A-10B illustrate an exemplary embodiment of real time stability of exosome molecular reference.

### DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definition

As used herein, the singular forms “a”, “an” and “the” include plural references unless the context clearly dictates otherwise.

It is noted that in this disclosure, terms such as “comprises”, “comprised”, “comprising”, “contains”, “containing” and the like are inclusive or open-ended and do not exclude additional, un-recited elements or method steps. Terms such as “consisting essentially of” and “consists essentially of” allow for the inclusion of additional ingredients or steps that do not materially affect the basic and novel characteristics of the claimed invention. The terms “consists of” and “consisting of” are close ended.

As used herein, the term “cancer” refers to any diseases involving an abnormal cell growth and includes all stages and all forms of the disease that affects any tissue, organ or cell in the body. The term includes all known cancers and neoplastic conditions, whether characterized as malignant, benign, soft tissue, or solid, and cancers of all stages and grades including pre- and post-metastatic cancers. In general, cancers can be categorized according to the tissue or organ from which the cancer is located or originated and morphology of cancerous tissues and cells. As used herein, cancer types include, acute lymphoblastic leukemia (ALL), acute myeloid leukemia, adrenocortical carcinoma, anal cancer, astrocytoma, childhood cerebellar or cerebral, basal-cell carcinoma, bile duct cancer, bladder cancer, bone tumor, brain cancer, breast cancer, Burkitt's lymphoma, cerebellar astrocytoma, cerebral astrocytoma/malignant glioma, cervical cancer, chronic lymphocytic leukemia, chronic myelogenous leukemia, colon cancer, emphysema, endometrial cancer, ependymoma, esophageal cancer, Ewing family of tumors, Ewing's sarcoma, gastric (stomach) cancer, glioma, head and neck cancer, heart cancer, Hodgkin lymphoma, islet cell carcinoma (endocrine pancreas), Kaposi sarcoma, kidney cancer (renal cell cancer), laryngeal cancer, leukaemia, liver cancer, lung cancer, medulloblastoma, melanoma, neuroblastoma, non-Hodgkin lymphoma, ovarian cancer, pancreatic cancer, pharyngeal cancer, prostate cancer, rectal cancer, renal cell carcinoma (kidney cancer), retinoblastoma, skin cancer, stomach cancer, supratentorial primitive neuroectodermal tumors, testicular cancer, throat cancer, thyroid cancer, vaginal cancer, visual pathway and hypothalamic glioma.

A “cell”, as used herein, can be prokaryotic or eukaryotic. A prokaryotic cell includes, for example, bacteria. A eukaryotic cell includes, for example, a fungus, a plant cell, and an animal cell. The types of an animal cell (e.g., a mammalian cell or a human cell) includes, for example, a cell from circulatory/immune system or organ (e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell); a cell from an endocrine system or organ (e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte)); a cell from a nervous system or organ (e.g., a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph)); a cell from a respiratory system or organ (e.g., a pneumocyte (a type I pneumocyte and a type II pneumocyte), a clara cell, a goblet cell, an alveolar macrophage); a cell from circular system or organ (e.g., myocardiocyte and pericyte); a cell from digestive system or organ (e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, a liver cell (e.g., a hepatocyte and Kupffer cell)); a cell from integumentary system or organ (e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell)), a muscle cell (e.g., myocyte), an adipocyte, a fibroblast, and a tendon cell), a cell from urinary system or organ (e.g., a podocyte, a juxtaglomerular cell, an intraglomerular mesangial cell, an extraglomerular mesangial cell, a kidney proximal tubule brush border cell, and a macula densa cell), and a cell from reproductive system or organ (e.g., a spermatozoon, a Sertoli cell, a leydig cell, an ovum, an oocyte). A cell can be normal, healthy cell; or a diseased or unhealthy cell (e.g., a cancer cell). A cell further includes a mammalian zygote or a stem cell which include an embryonic stem cell, a fetal stem cell, an induced pluripotent stem cell, and an adult stem cell. A stem cell is a cell that is capable of undergoing cycles of cell division while maintaining an undifferentiated state and differentiating into specialized cell types. A stem cell can be an omnipotent stem cell, a pluripotent stem cell, a multipotent stem cell, an oligopotent stem cell and a unipotent stem cell, any of which may be induced from a somatic cell. A stem cell may also include a cancer stem cell. A mammalian cell can be a rodent cell, e.g., a mouse, rat, hamster cell. A mammalian cell can be a lagomorpha cell, e.g., a rabbit cell. A mammalian cell can also be a primate cell, e.g., a human cell.

The term “genome editing enzyme” refers to an enzyme capable of altering or modifying the genetic sequence in a cell. Genome editing enzymes include, without limitation, site-specific nucleases (e.g., Cas9, ZFN, TALEN and meganuclease) and site-specific recombinases (e.g., Cre, FLP, lamda integrase, phiC31 integrase, Bxb1 integrase, gamma-delta resolvase, Tn3 resolvase and Gin invertase).

The term “kit” as used herein refers to a packaged combination of reagents in predetermined amounts with instructions for performing a therapeutics, or a diagnostic or detection assay.

The term “nucleic acid” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, a “nuclease” is an enzyme capable of cleaving the phosphodiester bonds between the nucleotide subunits of nucleic acids. A “site-specific nuclease” refers to a nuclease whose functioning depends on a specific nucleotide sequence. Typically, a site-specific nuclease recognizes and binds to a specific nucleotide sequence and cuts a phosphodiester bond within the nucleotide sequence. In certain embodiments, the double-strand break is generated by site-specific cleavage using a site-specific nuclease. Examples of site-specific nucleases include, without limitation, zinc finger nucleases (ZFNs), transcriptional activator-like effector nucleases (TALENs), meganuclease and CRISPR (clustered regularly interspaced short palindromic repeats)-associated (Cas) nucleases.

A site-specific nuclease typically contains a DNA-binding domain and a DNA-cleavage domain. For example, a ZFN contains a DNA binding domain that typically contains between three and six individual zinc finger repeats and a nuclease domain that consists of the FokI restriction enzyme that is responsible for the cleavage of DNA. The DNA binding domain of ZFN can recognize between 9 and 18 base pairs. In the example of a TALEN, which contains a TALE domain and a DNA cleavage domain, the TALE domain contains a repeated highly conserved 33-34 amino acid sequence with the exception of the 12^{th} and 13^{th} amino acids, whose variation shows a strong correlation with specific nucleotide recognition. For another example, Cas9, a typical Cas nuclease, is composed of an N-terminal recognition domain and two endonuclease domains (RuvC domain and HNH domain) at the C-terminus.

In general, a “protein” is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

As used herein, the term “recombinase” or “site-specific recombinase” refers to a family of highly specialized enzymes that promote DNA rearrangement between specific target sites (Greindley et al., 2006; Esposito, D., and Scocca, J. J., Nucleic Acids Research 25, 3605-3614 (1997); Nunes-Duby, S. E., et al, Nucleic Acids Research 26, 391-406 (1998); Stark, W. M., et al, Trends in Genetics 8, 432-439 (1992)). Virtually all site-specific recombinases can be categorized within one of two structurally and mechanistically distinct groups: the tyrosine (e.g., Cre, Flp, and the lambda integrase) or serine (e.g., phiC31 integrase, Bxb1 integrase, gamma-delta resolvase, Tn3 resolvase and Gin invertase) recombinases. Both recombinase families recognize target sites composed of two inversely repeated binding elements that flank a spacer sequence where DNA breakage and re-ligation occur. The recombination process requires concomitant binding of two recombinase monomers to each target site: two DNA-bound dimers (a tetramer) then join to form a synaptic complex, leading to crossover and strand exchange.

The term “subject” or “individual” or “animal” or “patient” as used herein refers to human or non-human animal, including a mammal or a primate, in need of diagnosis, prognosis, amelioration, prevention and/or treatment of a disease or disorder such as viral infection or tumor. Mammalian subjects include humans, domestic animals, farm animals, and zoo, sports, or pet animals such as dogs, cats, guinea pigs, rabbits, rats, mice, horses, swine, cows, bears, and so on.

In the context of formation of a CRISPR complex, “target” refers to a guide sequence (that is, gRNA) designed to have complementarity to a genomic region (that is, a target sequence), where hybridization between the genomic region and a guide RNA promotes the formation of a CRISPR complex. The terms “complementarity” or “complementary” are used in reference to polynucleotides (i.e., a sequence of nucleotides) related by the base-pairing rules. Complementarity may be “partial,” in which only some of the nucleic acids' bases are matched according to the base pairing rules (e.g., 5, 6, 7, 8, 9, 10 out of 10 being 50%, 60%, 70%, 80%, 90%, and 100% complementary), or there may be “complete” or “total” complementarity between the nucleic acids. The degree of complementarity between nucleic acid strands has significant effects on the efficiency and strength of their hybridization to one another.

Engineered Cell Line

The present disclosure in one aspect relates to engineered cell lines that contain specific mutations or transgenes and the exosomes secreted from these cell lines.

In certain embodiments, the engineered cell lines described herein are generated using genome editing technology, e.g., by using genome editing enzymes. In certain embodiments, genome editing enzymes include, without limitation, site-specific nucleases (e.g., Cas9, ZFN, TALEN and meganuclease) and site-specific recombinases (e.g., Cre, FLP, lamda integrase, phiC31 integrase, Bxb1 integrase, gamma-delta resolvase, Tn3 resolvase and Gin invertase).

CRISPR (Clustered Regularly Interspaced Short Palindromic Repeats)/Cas system was originally found as transcripts and other elements in the prokaryotic cells involved in the expression of or directing the activity of CRISPR-associated (“Cas”) genes, including sequences encoding a Cas nuclease that cleaves the nucleic acid sequence and generates double strand break (DSB), a guide sequence, a trans-activating CRISPR (tracr) sequence, a tracr-mate sequence, or other sequences and transcripts from a CRISPR locus. In eukaryotic cells, the CRISPR/Cas system comprises a CRISPR-associated nuclease and a small guide RNA. The target DNA sequence (the protospacer) contains a “protospacer-adjacent motif” (PAM), a short DNA sequence recognized by the particular Cas protein being used. In certain embodiments, the CRISPR system comprises CRISPR/Cas system of type I, type II, and type III, which comprises protein Cas3, Cas9 and Cas10, respectively.

The RNA-guided endonuclease Cas9 is a component of the type II CRISPR system widely utilized generate gene-specific knockouts in a variety of model systems. In one embodiment of the present disclosure, the CRISPR/Cas nuclease is a “sequence-specific nuclease”. Introduction of ectopic expression of Cas9 and a single guide RNA (gRNA) is sufficient to lead to the formation of double-strand breaks (DSBs) at a specific genomic region of interest, which leads to an indel via non-homologous end joining (NHEJ) pathway. Indels often result in frameshift mutations, except when the number of inserted/deleted nucleotides is a multiple of 3.

Along with Cas endonuclease, CRISPR experiments require the introduction of a guide RNA containing an approximately 15 to 30 base sequence specific to a target nucleic acid (e.g., DNA). A gRNA designed to target a genomic region of interest, for example, a particular exon encoding a functional domain of a protein, will generate a mutation in each gene that encodes the protein. The resulted modified genomic region may comprise one or more variants, each of which is different in the mutation. For example, the mutation will result in a modified genomic region with a desired modification, and/or a modified genomic region with an undesired modification. This approach has been widely utilized to generate gene-specific knockouts in a variety of model systems. In certain embodiments, a gRNA has a length of 15, 16, 17, 18, 19, 20, 21, 22, 23, 24, 25, 26, 27, 28, 29 or 30 nucleotides. gRNA can be delivered into a eukaryotic cell or a prokaryotic cell as RNA or by transfection with a vector (e.g., plasmid) having a gRNA-coding sequence operably linked to a promoter.

In certain embodiments, the Cas nuclease and the gRNA are derived from the same species. In certain embodiments, the Cas nuclease is derived from, for example, Staphylococcus aureus, Staphylococcus epidermidis, Staphylococcus sciuri, Pseudomonas aeruginosa, Enterococcus faecium, Enterococcus faecalis, Escherichia coli, Klebsiella pneumoniae, Streptococcus pneumoniae, Streptococcus pyrogenes, Lactobacillus bulgaricus, Streptococcus thermophilus Vibrio cholera, Achromobacter xylosoxidans, Burkholderia cepacia, Citrobacter diversus, Citrobacter freundii, Micrococcus leuteus, Proteus mirabilis, Proteus vulgaris, Staphylococcus lugdunegis, Salmonella typhi, Streptococcus Group A, Streptococcus Group B, S. marcescens, Enterobacter cloacae, Bacillus anthracis, Bordetella pertussis, Clostridium sp., Clostridium botulinum, Clostridium tetani, Corynebacterium diphtheria, Moraxalla (Brauhamella) catarrhalis, Shigella spp., Haemophilus influenza, Stenotrophomonas maltophili, Pseudomonas perolens, Pseuomonas fragi, Bacteroides fragilis, Fusobacterium sp. Veillonella sp., Yersinia pestis, and Yersinia pseudotuberculosis.

A gRNA can be designed using any known software in the art, such as Target Finder, E-CRISPR, CasFinder, and CRISPR Optimal Target Finder.

In certain embodiments, the composition described herein comprises a nucleic acid encoding the Cas nuclease or the gRNA, wherein the nucleic acid is contained in a vector. In some embodiments, the composition comprises Cas nuclease protein and a DNA encoding the gRNA. In some embodiments, the composition comprises a first nucleic acid encoding the Cas nuclease and a second nucleic acid encoding the gRNA, whereas the first and the second nucleic acids are contained in one vector. In some embodiment, the first and the second nucleic acids are contained in two separate vectors. In some embodiments, at least one vector is a viral vector. In certain embodiments, the vector is AAV vector.

A zinc finger nuclease (ZFN) is an artificial restriction enzyme generated by fusing a zinc finger DNA-binding domain to a DNA-cleavage domain. Zinc finger domain can be engineered to target specific desired DNA sequences, which directs the zinc finger nucleases to cleave the target DNA sequences. Typically, a zinc finger DNA-binding domain contains three to six individual zinc finger repeats and can recognize between 9 and 18 base pairs. Each zinc finger repeat typically includes approximately 30 amino acids and comprises a ββα-fold stabilized by a zinc ion. Adjacent zinc finger repeats arranged in tandem are joined together by linker sequences. Various strategies have been developed to engineer zinc finger domains to bind desired sequences, including both “modular assembly” and selection strategies that employ either phage display or cellular selection systems (Pabo C O et al., “Design and Selection of Novel Cys2His2 Zinc Finger Proteins” Annu. Rev. Biochem. (2001) 70:313-40). The most straightforward method to generate new zinc-finger DNA-binding domains is to combine smaller zinc-finger repeats of known specificity. The most common modular assembly process involves combining three separate zinc finger repeats that can each recognize a 3 base pair DNA sequence to generate a 3-finger array that can recognize a 9 base pair target site. Other procedures can utilize either 1-finger or 2-finger modules to generate zinc-finger arrays with six or more individual zinc finger repeats. Alternatively, selection methods have been used to generate zinc-finger DNA-binding domains capable of targeting desired sequences. Initial selection efforts utilized phage display to select proteins that bound a given DNA target from a large pool of partially randomized zinc-finger domains. More recent efforts have utilized yeast one-hybrid systems, bacterial one-hybrid and two-hybrid systems, and mammalian cells. A promising new method to select novel zinc-finger arrays utilizes a bacterial two-hybrid system that combines pre-selected pools of individual zinc finger repeats that were each selected to bind a given triplet and then utilizes a second round of selection to obtain 3-finger repeats capable of binding a desired 9-bp sequence (Maeder M L, et al., “Rapid ‘open-source’ engineering of customized zinc-finger nucleases for highly efficient gene modification”. Mol. Cell (2008) 31(2): 294-301). The non-specific cleavage domain from the type II restriction endonuclease FokI is typically used as the cleavage domain in ZFNs. This cleavage domain must dimerize in order to cleave DNA and thus a pair of ZFNs are required to target non-palindromic DNA sites. Standard ZFNs fuse the cleavage domain to the C-terminus of each zinc finger domain. In order to allow the two cleavage domains to dimerize and cleave DNA, the two individual ZFNs must bind opposite strands of DNA with their C-termini a certain distance apart. The most commonly used linker sequences between the zinc finger domain and the cleavage domain requires the 5′ edge of each binding site to be separated by 5 to 7 bp.

A transcription activator-like effector nuclease (TALEN) is an artificial restriction enzyme made by fusing a transcription activator-like effector (TALE) DNA-binding domain to a DNA cleavage domain (e.g., a nuclease domain), which can be engineered to cut specific sequences. TALEs are proteins that are secreted by Xanthomonas bacteria via their type III secretion system when they infect plants. TALE DNA-binding domain contains a repeated highly conserved 33-34 amino acid sequence with divergent 12th and 13th amino acids, which are highly variable and show a strong correlation with specific nucleotide recognition. The relationship between amino acid sequence and DNA recognition allows for the engineering of specific DNA-binding domains by selecting a combination of repeat segments containing the appropriate variable amino acids. The non-specific DNA cleavage domain from the end of the FokI endonuclease can be used to construct TALEN. The FokI domain functions as a dimer, requiring two constructs with unique DNA binding domains for sites in the target genome with proper orientation and spacing. See Boch, Jens “TALEs of genome targeting” Nature Biotechnology (2011) 29: 135-6; Boch, Jens et al., “Breaking the Code of DNA Binding Specificity of TAL-Type III Effectors” Science (2009) 326: 1509-12; Moscou M J and Bogdanove A J “A Simple Cipher Governs DNA Recognition by TAL Effectors” Science (2009) 326 (5959): 1501; Juillerat A et al., “Optimized tuning of TALEN specificity using non-conventional RVDs” Scientific Reports (2015) 5: 8150; Christian et al., “Targeting DNA Double-Strand Breaks with TAL Effector Nucleases” Genetics (2010) 186 (2): 757-61; Li et al., “TAL nucleases (TALNs): hybrid proteins composed of TAL effectors and FokI DNA-cleavage domain” Nucleic Acids Research (2010) 39: 1-14.

Site-specific recombinases refer to a family of enzymes that mediate the site-specific recombination between specific DNA sequences recognized by the enzymes. Examples of site-specific recombinase include, without limitation, Cre recombinase, Flp recombinase, the lambda integrase, gamma-delta resolvase, Tn3 resolvase, Sin resolvase, Gin invertase, Hin invertase, Tn5044 resolvase, Tn3 transposase, sleeping beauty transposase, IS607 transposase, Bxb1 integrase, wBeta integrase, BL3 integrase, phiR4 integrase, A118 integrase, TG1 integrase, MR11 integrase, phi370 integrase, SPBc integrase, SV1 integrase, TP901-1 integrase, phiRV integrase, FC1 integrase, K38 integrase, phiBT1 integrase and phiC31 integrase.

The engineered cell line described herein can contain mutation in any desired gene or contain any desired transgene. In certain embodiments, the engineered cell lines described herein contain a mutation in a cancer gene. Non-limiting examples of cancer genes include, EGFR, KRAS, BRAF, PIK3CA, AKT1, NRAS, HRAS, TP53, BRCA1, BRCA2, JAK2, RB1, PTEN, CTNNB1, APC, FLT3, KIT, ESR1, ERBB2, MAP2K1, FGR3, IDH1, IDH2, ATM, PIK3R1, FGFR2, PDGFRA, ABL1, FGFR1, GNA11, NOTCH1, GNAQ, GNAS, CDH1, CD2, MLH1, MET, ALK, RET, SMAD4, ROS1, BARD1, BRIP1, FBXW7, NBN, STK11, EML4-ALK, CD74-ROS1, KDR, ALK, RAF1, MTOR, CHEK2, PLE, POLD1, KIF5B-ALK, CCDC6-RET, BCR-ABL1, CD74-ROS1.

Examples of mutations, without limitation, include EGFR-G719S, EGFR-G719C, EGFR-G19A, EGFR-L747_S752del, EGFR-L747_P753>S, EGFR-E746_S752>D, EGFR-L747_P753>Q, EGFR-E746_S752>A, EGFR-E746_S752>V, EGFR-E746_T751del, EGFR-E746_T751>A, EGFR-L747_T751del, EGFR-E746_T751>V, EGFR-E746_T751>I, EGFR-K745_E749del, EGFR-E746_A750del, EGFR-L747_T751>P, EGFR-L747_T751>S, EGFR-L747_T751>Q, EGFR-L747_A750>P, EGFR-L747_E749del, EGFR-E746_A750>IP, EGFR-V769_D770insASV, EGFR-S768I, EGFR-D770_N771insG, EGFR-H773_V774insH, EGFR-D761Y, EGFR-861Q, EGFR-C797S/T790M, EGFR-C326Y, EGFR-D761Y, EGFR-L747S, EGFR-S784F, EGFR-Q787R, EGFR-N826S, EGFR-T854A, EGFR-V843I, EGFR-V843I/L858R, EGFR-G719A/T790M, EGFR-D761Y/T790M EGFR-L747S/T790M, EGFR-T854A/T790M, EGFR-G598V, EGFR-E709K, EGFR-E709A, EGFR-L833V, EGFR-S492R, EGFR-E884K, EGFR-I491M, EGFR-S464L, KRAS-G12C, KRAS-G12S, KRAS-G12R, KRAS-G12V, KRAS-G12D, KRAS-G12A, KRAS-G13C, KRAS-G13S, KRAS-G13R, KRAS-G13D, KRAS-G13A, KRAS-G13V, BRAF-V600K, BRAF-V600R, BRAF-V600E, BRAF-V600E, BRAF-V600D, BRAF-V600M, BRAF-V600G, PIK3CA-H1047R, PIK3CA-H1047L, PIK3CA-E542K, PIK3CA-E545K, PIK3CA-E545G, PIK3CA-E545A, PIK3CA-Q546K, PIK3CA-R88Q, PIK3CA-N345K, PIK3CA-C420R, KIT-W557_K558del, KIT-W557G, KIT-W557_V559>F, KIT-W557R, KIT-D816V, KIT-D816Y, KIT-D816H, KIT-V559D, KIT-V559A, KIT-V560D, AKT1-E17K, KRAS-Q61H, KRAS-A146T, KRAS-K117N, KRAS-A146V, KRAS-Q61L, KRAS-A59T, NRAS-G12D, NRAS-G12S, NRAS-G12C, NRAS-G13D, NRAS-G13R, NRAS-G13V, NRAS-Q61R, NRAS-Q61K, NRAS-Q61L, NRAS-A146T, HRAS-G12S, HRAS-G12V, HRAS-G12D, HRAS-G13R, HRAS-Q61K, HRAS-Q61R, HRAS-Q61L, HRAS-G12C, HRAS-G13V, HRAS-G13D, HRAS-G13S, HRAS-Q61H, EML4-ALK (E13;A20, E6;A20, E20;A20), KIF5B-RET (K15;R12, K16;R12, K16;R12, K22;R12), CD74-ROS1 (C6;R34), EZR-ROS1 (E10;R34), TP53-R175H, TP53-R175G, TP53-R175L, TP53-Y220C, TP53-C242Y, TP53-G245S, TP53-G245D, TP53-G245V, TP53-G245C, TP53-R248Q, TP53-R248W, TP53-R248L, TP53-R249S, TP53-R249W, TP53-R249M, TP53-R249G, TP53-R273H, TP53-R273C, TP53-R273L, TP53-D281G, TP53-R282W, TP53-R282G, TP53-R282Q, TP53-K382fs*>12, T53-R158H, T53-R158L, T53-R158C, T53-R158G, T53-V157F, T53-V157G, T53-V1571, T53-H179R, T53-G154V, T53-G154S, T53-Y163C, T53-Y163N, T53-Y163H, T53-V173L, T53-V173M, T53-C176F, T53-C176Y, T53-Q192*, T53-Y205C, T53-Y205D, T53-R213*, T53-R213L, T53-R213Q, T53-H214R, T53-Y234C, T53-Y234H, T53-M237I, T53-C238Y, T53-C238F, T53-C242F, T53-E286K, T53-E298*, BRCA1-R1835*, BRCA1-E23fs*17, BRCA1-Q1756fs*74 BRCA1-C61G p.L292*, BRCA1-E402*, BRCA1-V1713*, BRCA1-V1713A, BRCA1-K1183R, BRCA1-P871L, BRCA2-S1982fs*22, BRCA2-N372H, BRCA2-R2645fs*3, IDH1-R132H, IDH1-R132L, IDH1-R132C, IDH1-R132G, IDH1-R132S, IDH1-R132S, IDH1-R132H, IDH2-R140Q, IDH2-R140W, IDH2-R140L, IDH2-R172K, IDH2-R172M, IDH2-R172S, IDH2-R172W, IDH2-R172G, ALK-F1174L, ALK-F1174C, ALK-R1275Q, ALK-R1275L, ALK-L1196M, ALK-T1151_L1152insT, ALK-L1152R, ALK-C1156Y, RET-M918T, RET-C634R, RET-C634Y, SMAD4-R361C SMAD4-R361H, SMAD4-D351H, SMAD4-P356L, SMAD4-P356S, ROS1-E402K, ROS1-E1642K, ROS1-E1541D, JAK2-V617F, JAK2-V615L, JAK2-V6171, RB1-R579*, RB1-R30*, RB1-R251*, RB1-R661W, PTEN-R130G, PTEN-R130Q, PTEN-R130L, PTEN-R130*, PTEN-R173C, PTEN-173H, PTEN-R233*, PTEN-A126G, CTNNB1-T41A, CTNNB1-S45F, CTNNB1-S45P, CTNNB1-S37F, CTNNB1-S33C, CTNNB1-S33Y, CTNNB1-S37C, CTNNB1-T41I, CTNNB1-S45del, APC-R1450*, APC-T1556fs*3, APC-E1309fs*4, APC-Q1367*, APC-S1465fs*3, APC-T1556fs*9, APC-R213*, APC-R876*, FLT3-D835Y, FLT3-D835V, FLT3-D835H, FLT3-Y597_E598insDYVDFREY, FLT3-D600_L601insFREYEYD, FLT3-I836delI, ESR1-K303R, ESR1-D538G, ESR1-Y537S, ESR1-Y537N, ESR1-Y537C, ERBB2-A775_G776insYVMA, ERBB2-V777L, ERBB2-G776>VC, MET-H1256R, MET-Y1248H, MET-H1124Y, MET-H1124D, MET-D999Y, MET-Q348R, PDGFRA-N659K, PDGFRA-N659K, PDGFRA-N659Y, PDGFRA-D1071N, FGFR2-A264T, FGFR2-K292M, FGFR2-G302R, FGFR2-S436F, FBXW7-R393*, FBXW7-G459E, FBXW7-R479L, FBXW7-R479P, FBXW7-S582L, GNAS-A249T, GNAS-R232C, GNAS-D229G, GNAS-R228H, PIK3R1-R348*, PIK3R1-D560G, PIK3R1-D560Y, PIK3R1-N564D, PIK3R1-K567E, PIK3R1-L573P, PIK3R1-R574I, PIK3R1-T576fs*26, PIK3R1-T576del, ATM-R337C, ATM-R337H, ATM-C2337R, ATM-R3008C, ATM-R3008H, FGR3-S249C, FGR3-R248C, FGR3-Y373C, FGR3-G370C, FGR3-S371C, FGR3-G380R, FGR3-A391E, FGR3-K650E, FGR3-K650Q, FGR3-K650M, FGR3-K650T, ABL1-Y253H, ABL1-E255K, ABL1-E255V, ABL1-T315I, ABL1-M351T, GNA11-Q209L, GNA11-Q209R, GNA11-R183C, NOTCH1-P2514fs*4, NOTCH1-T311P, NOTCH1-L1574P, NOTCH1-V1578delV, NOTCH1-L1593P, NOTCH1-R1598P, NOTCH1-L1600P, NOTCH1-L1678P, NOTCH1-D1698D, GNAQ-Q209L, GNAQ-Q209P, GNAQ-Q209L, GNAQ-Q209R, GNAQ-R183Q, GNAQ-T96S, CDH1-P126fs*89, CDH1-D254Y, CDH1-R732Q, CDX2-V306fs*2, MLH1-L323M, MLH1-V384D, MLH1-I219V, BARD1-R378S, BRIP1-S919P, NBN-E185Q, KDR-V2971, KDR-Q472H, KDR-R1032Q, APC-p.S1364fs*11, FGR1-N546K, FGR1-K656E, STK11-F354L, STK11-Q37*, STK11-P281L, STK11-Q170*, STK11-G171S, POLD1-S478N, POLE-V474I, POLE-L424V, POLE-P286R, POLE-V411L, RAF1-S257L, RAF1-S259F, MTOR-S2215Y, MTOR-S2215F, MTOR-E1799K, CHEK2-T367fs*15, CHEK2-R145W, CHEK2-Y390C, CHEK2-K373E,

In certain embodiments, the transgene that can be introduced into the engineered cell line encodes a microRNA, a non-coding RNA, an mRNA, a tRNA, an rRNA, siRNA or an shRNA. Examples of microRNA, without limitation, include miR-9, miR-629, miR-141, miR-671-3p, miR-491, miR-182, miR-125a-3p, miR-324-5p, miR-148b, and miR-222.

Generation of Exosome and Exosome RNA

The engineered cell lines described herein can be used to generate exosomes that contain RNA comprising desired mutation or transgene. The exosome can further be used to generate RNA that comprises the desired mutation or transgene.

Exosomes are small vesicles that are released into the extracellular environment from a variety of different cells such as but not limited to, cells that originate from, or are derived from, the ectoderm, endoderm, or mesoderm including any such cells that have undergone genetic, environmental, and/or any other variations or alterations (e.g. Tumor cells or cells with genetic mutations). An exosome is typically created intracellularly when a segment of the cell membrane spontaneously invaginates and is ultimately exocytosed (see for example, Keller et al., Immunol. Lett. 107 (2): 102-8 (2006)). Exosomes can have, but not be limited to, a diameter of greater than about 10, 20, or 30 nm. They can have a diameter of about 30-1000 nm, about 30-800 nm, about 30-200 nm, or about 30-100 nm. In some embodiments, the exosomes can have, but not be limited to, a diameter of less than about 10,000 nm, 1000 nm, 800 nm, 500 nm, 200 nm, 100 nm or 50 nm.

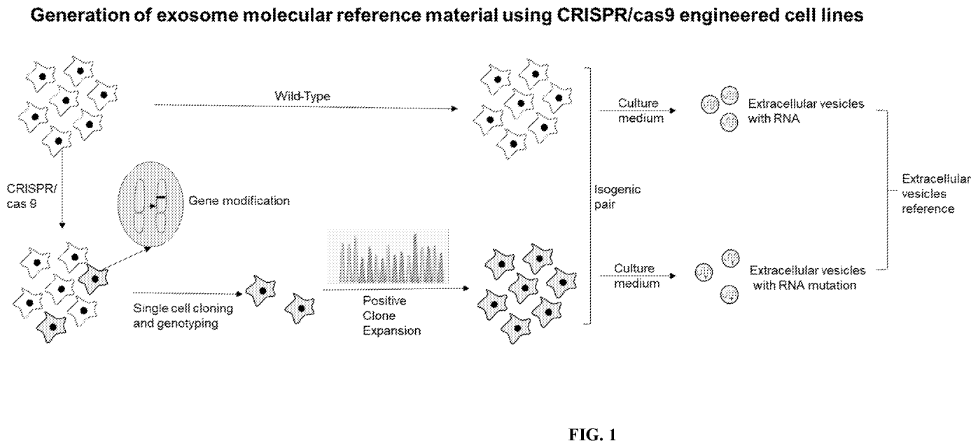

FIG. 1 illustrates an exemplary embodiment of generating exosomes from the engineered cell lines described herein. Referring to FIG. 1, a population of cells is modified with CRISPR/Cas9 to introduce a gene modification in at least some of the cells in the population. The cells comprising the gene modification are then identified using single cell cloning and genotyping. The identified cells are expanded and cultured in suitable medium, which produces exosomes with RNA transcribed from the modified gene, i.e. mutated RNA.

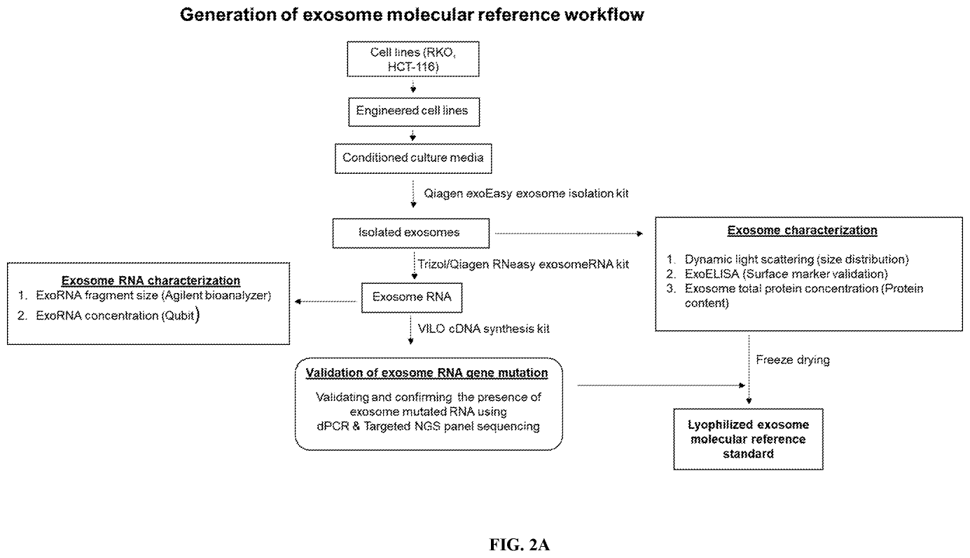

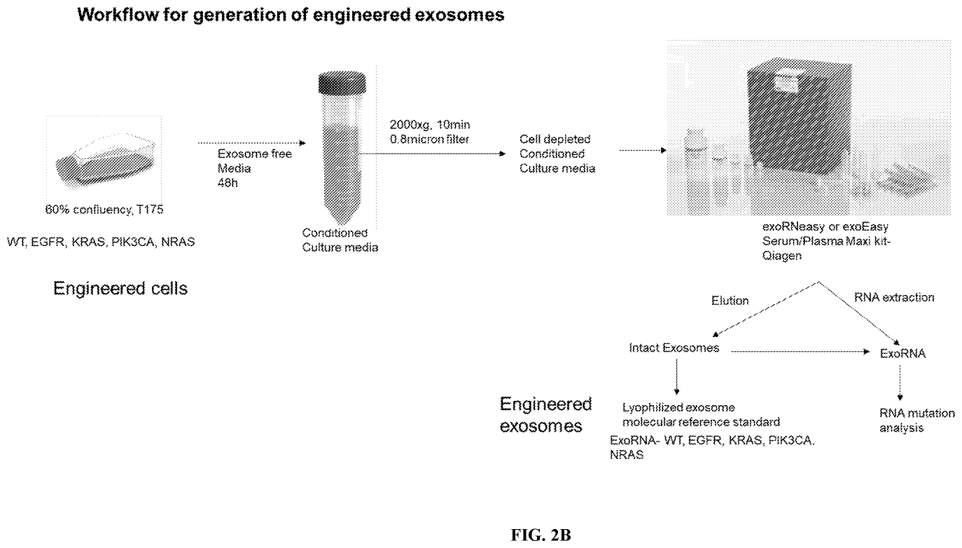

FIGS. 2A and 2B illustrate an exemplary workflow of generation of exosome and exosome RNA from the engineered cell lines described herein. Referring FIG. 2A, cell lines, e.g., RKO and HCT-116 are modified with gene editing technology to generate engineered cell lines that contains desired gene modification. The conditioned culture medium of the engineered cell lines is collected, and cells in the culture medium are removed by centrifuge and filter (see FIG. 2B). The cell-depleted conditioned culture medium is then used to isolate exosomes containing mutated RNA with Qiagen exoEasy exosome isolation kit. The isolated exosomes are used to isolate exosome RNA with Trizol/Qiagen exoRNeasy exosomeRNA kit. The isolated exosomes are characterized by dynamic light scattering to assay the size distribution, by ExoELISA to detect surface marker, and by exosome total protein concentration to assay the protein content. The isolated exosome RNA is characterized to assay the fragment size and RNA concentration. The isolated exosome RNA is also validated to contain desired mutated RNA.

Use of Exosome and Exosome RNA

The exosome and exosome RNA described herein have a variety of applications.

Exosomes can be used for detecting biomarkers for diagnostic, therapy-related or prognostic methods to identify phenotypes, such as a condition or disease, for example, the stage or progression of a disease (e.g. U.S. Pat. No. 7,897,356 to Klass et al.). In these methods, reference materials are needed to ensure that the exosomes or exoRNA are properly isolated and detected from the sample of a subject. In certain embodiments, the exosome and exosome RNA described herein can be used as reference materials in the detection of exosomes, e.g., exosomes isolated from patient biofluids. In certain embodiments, the exosome and exosome RNA described herein can be used in quality control and in a proficiency panel. Therefore, in one aspect, the present disclosure provides a kit comprising the exosome or exosome RNA described herein. In certain embodiments, the kit may further comprise reagents for isolating exosome or isolating RNA from exosome. In certain embodiments, the kit may further comprise reagents for detecting a mutation or polymorphism in exosome RNA. In certain embodiments, exosome RNA mutation can be employed to estimate the limit-of-detection (LOD) assessment. In particular, to validate exosome-based clinical assay for understanding lot-to-lot variation, cross-site performance, and inter-operator reproducibility. In another aspect, the present disclosure provides a method of diagnosing a disease based on analyzing exosomes or exosome RNA isolated from patient biofluids and using the exosome and exosome RNA described herein as reference material.

In certain embodiments, the exosome described herein may be used as a therapeutic delivery device, e.g., for delivering specific RNA, e.g., microRNA, siRNA, non-coding RNA, mRNA, tRNA, rRNA and shRNA. Therefore, the present disclosure in another aspect provides a pharmaceutical composition comprising the exosome or exosome RNA described herein, e.g., mutated RNA. In another aspect, the present disclosure provides a method for treating a disease in a subject by administering to the subject a therapeutically effective amount of the exosome described herein.

### Example 1

This example illustrates the exosomes reference material generated from engineered cell lines.

Methods: CRISPR/Cas9 targeting reagents were transfected into either HCT116 or RKO cell line. Exosomes were produced by culturing the engineered cells in exosome-free serum culture media. Exosomes were then isolated from ExoEasy kit (Qiagen). For genetic analysis, exo-RNA was isolated from the exosomes using trizol/membrane filter in ExoRNeasy kit. Allelic rare mutations in RNA were verified by digital PCR and validated by targeted NGS.

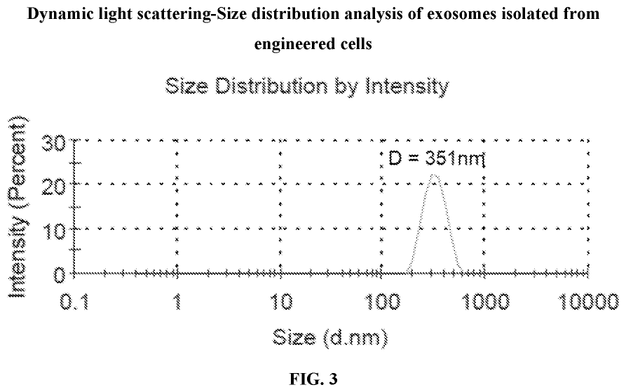

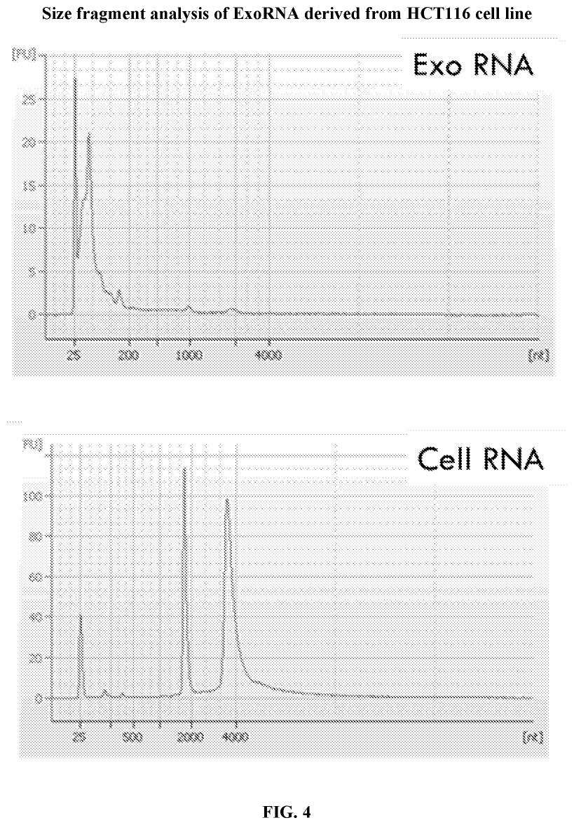

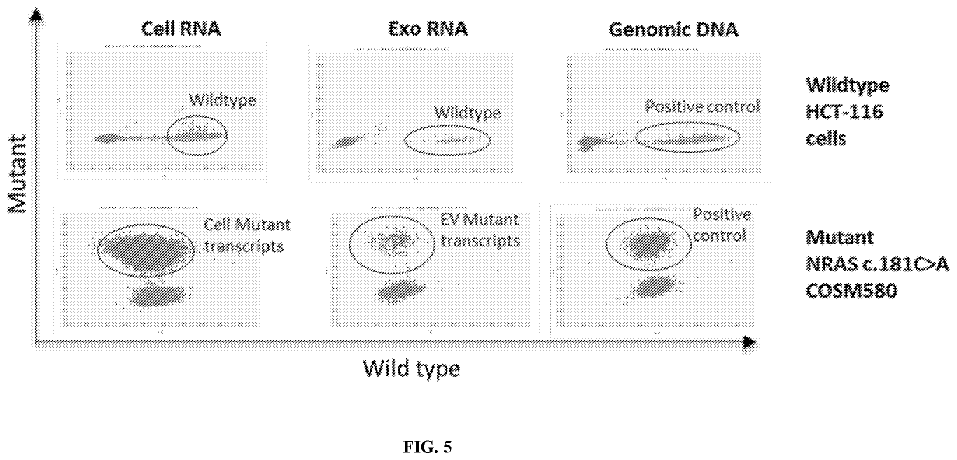

Results: The engineered cells that are homozygous of mutation were identified by Sanger Sequencing. As illustrated in FIG. 3, the exosomes isolated from the engineered cells had sizes centered at 351 nm in diameter using dynamic light scattering assay. The ExoRNA derived from the engineered cells had a fragmentation profile centered at approximately 25-200 bp (see FIG. 4). The mutant transcripts in the engineered cells and in the exosomes derived from the engineered cells were validated by digital PCR (dPCR) (see FIG. 5). Targeted variants including EGFR-T790M, EGFR-L858R, PIK3CA-E45K, NRAS-Q61K showed measurable copies of ExoRNA and cell-RNA from engineered cells (see Table 1). Digital PCR verified variants in ExoRNA was also detected and confirmed by NGS at 100% mutation frequency.

TABLE 1
dPCR validation of exosome mutant transcript levels in multiple genes

| | | | Cell RNA mutant | ExoRNA mutant | Cell RNA WT | ExoRNA WT |
|---|---|---|---|---|---|---|
| | Nucleotide | Amino acid | copy/ng | copies/10 ng | copies/ng | copies/ng |
| Gene | mutation | change | total RNA | total RNA | total RNA | total RNA |
| EGFR | 2155G > A | G719S | 110 | 60 | | |
| EGFR | 2369C > T | T790M | 123 | 20 | | |
| EGFR | 2573T > G | L858R | 2.5 | 5 | | |
| KRAS | 35G > A | G12D | 21 | 2 | | |
| PIK3CA | c.1633G > A | p.E45K | 84 | 180 | | |
| NRAS | c.181C > A | pQ61K | 1184 | 130 | | |
| Wild type | | | | | 460 | 21 |

### Example 2

This example illustrates the stability of the exosome RNA generated from the engineered cell line.

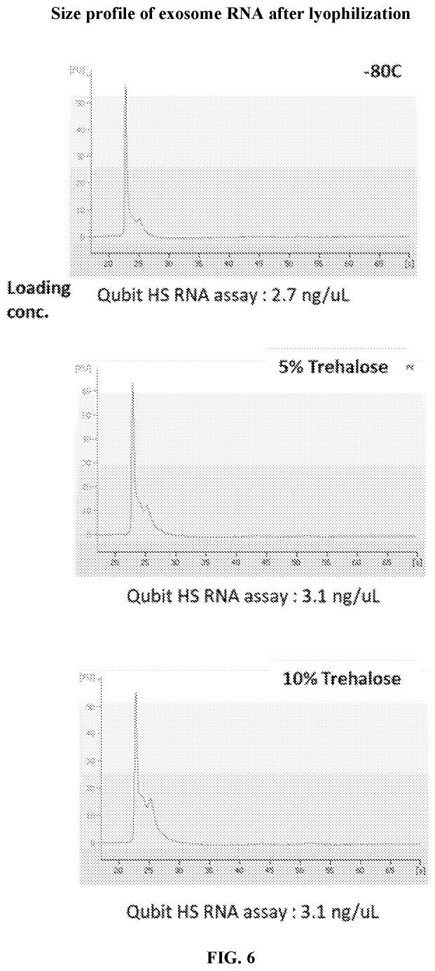

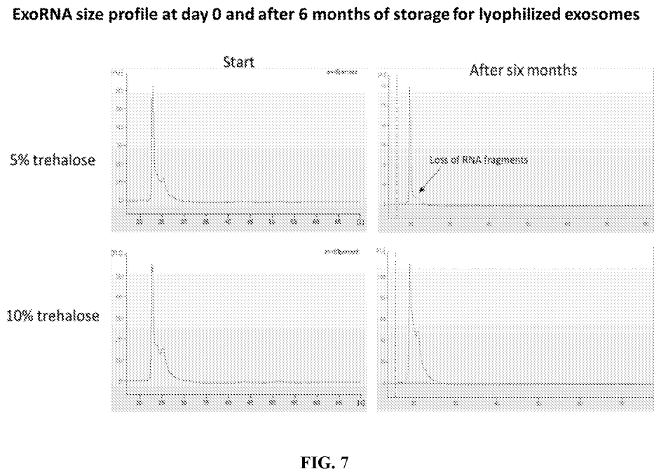

The exosome RNA generated from the engineered cell line as described in Example 1 were lyophilized in the presence of 5% or 10% Trehalose. After 6 months storage, the lyophilized exosome RNA was analyzed for size profile and measurable copies of ExoRNA. As illustrated in FIG. 6, exosome RNA lyophilized in the presence of Trehalose had similar size profile as the exosome RNA stored at −80° C. As illustrated in FIG. 7, the exosome RNA lyophilized in 5% Trehalose had increased loss of RNA fragments as compared to the exosome RNA lyophilized in 10% Trehalose after 6 months storage.

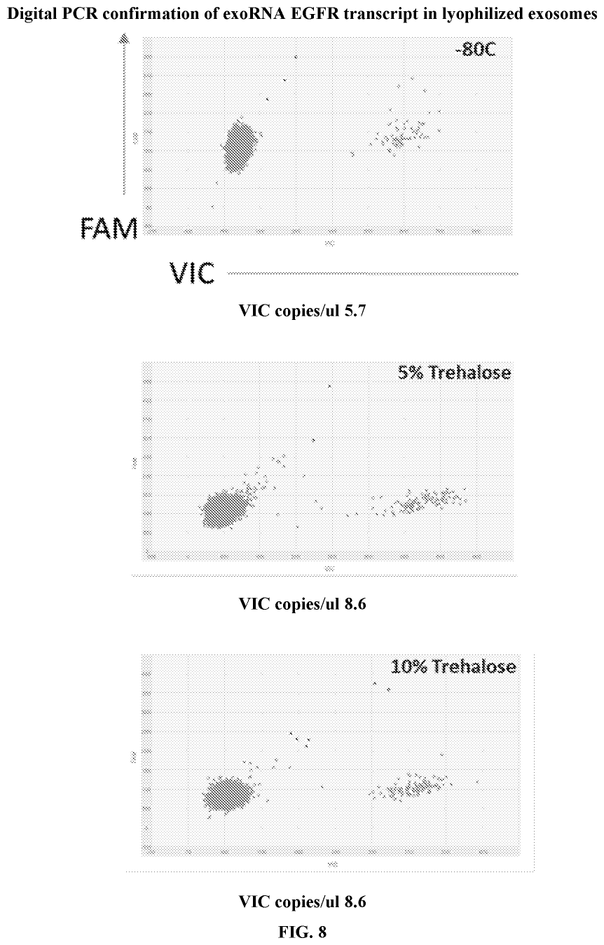

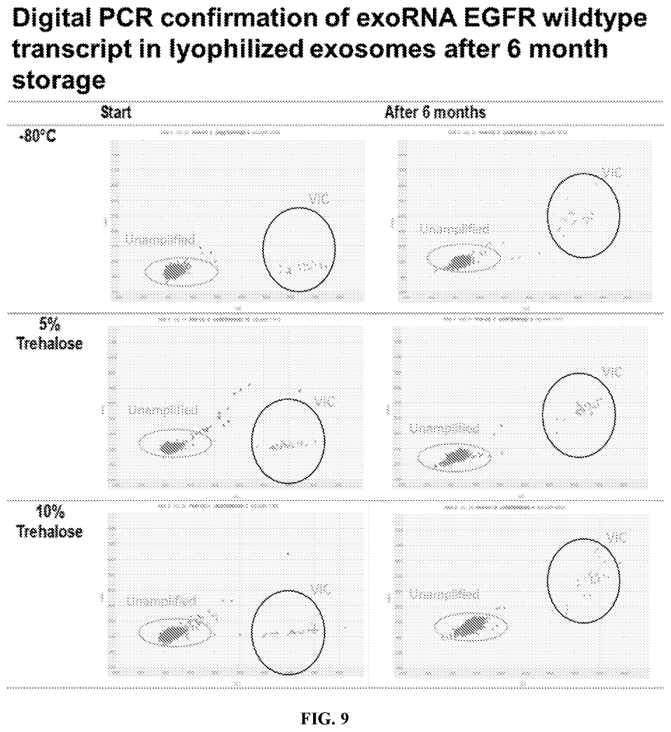

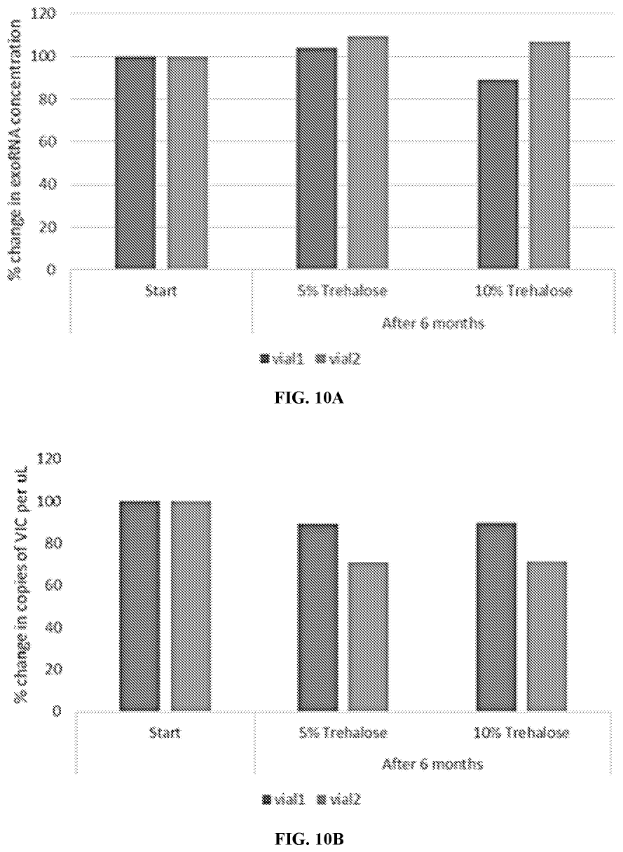

The presence of mutant transcript in the exosome RNA derived from the lyophilized exosomes after 6 months storage was confirmed by ddPCR (see FIG. 8 and FIG. 9). As illustrated in FIG. 10A, 6 months storage of the lyophilized exosome RNA did not change in concentration. As shown in FIG. 10B, after 6 months storage, the measurable copies of the mutant RNA in exosome did not significantly change.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

1. A method for producing a panel of exosome, the method comprising:
generating a plurality of cells, each comprising a mutation of a respective cancer gene by using a genome editing enzyme;
culturing the plurality of cells in a medium that allows each of the plurality of cells to secrete to the medium an exosome containing an RNA transcribed from the respective cancer gene and comprising the mutation; and
collecting the medium that contains the exosome, thereby generating a panel of exosomes comprising a panel of cancer specific mutations.

2. The method of claim 1, wherein the plurality of cells is generated from a cell line.

3. The method of claim 2, wherein the cell line is HCT116 or RKO.

4. The method of claim 1, wherein the plurality of cells is generated from a stem cell.

5. The method of claim 4, wherein the stem cell is an induced pluripotent stem cell (iPSC).

6. The method of claim 1, wherein the genome editing enzyme is a CRISPR/Cas nuclease, a zinc-finger nuclease (ZFN) or a transcription activator-like effector nuclease (TALEN).

7. The method of claim 1, wherein the plurality of cells is homozygous in the mutation of the respective cancer gene.

8. The method of claim 1, wherein the plurality of cells is heterozygous in the mutation of the respective cancer gene.

9. (canceled)

10. The method of claim 1, wherein the cancer gene is selected from the group consisting of EGFR, KRAS, BRAF, PIK3CA, AKT1, NRAS, HRAS, TP53, BRCA1, BRCA2, JAK2, RB1, PTEN, CTNNB1, APC, FLT3, KIT, ESR1, ERBB2, MAP2K1, FGR3, IDH1, IDH2, ATM, PIK3R1, FGFR2, PDGFRA, ABL1, FGFR1, GNA11, NOTCH1, GNAQ, GNAS, CDH1, CD2, MEH1, MET, ALK, RET, SMAD4, ROS1, BARD1, BRIP1, FBXW7, NBN, STK11, KIT, EML4-ALK, CD74-ROS1, KDR, APC, ALK, RAF1, MTOR, ATM, CHEK2, AKT1, FGFR2, PLE, POLD1, KIF5B-ALK, CCDC6-RET, BCR-ABL1, and CD74-ROS1.

11. The method of claim 1, wherein the mutation is a point mutation, an insertion, a deletion or a gene fusion.

12. The method of claim 1, wherein the mutation is selected from the group consisting of EGFR-T790M, EGFR-L858R, EGFR-V769_D770insASV, EGFR-E746_A750del, EGFR-E746_A750delELREA, EGFR-G719S, EGFR-L747_P753>S, EGFR-D761Y, EGFR-861Q, EGFR-S768I, EGFR-G719S, EGFR-C797S, KIT-D816V, PIK3CA-E45K, PIK3CA-H1047L, NRAS-Q61K, KRAS-G12D, BRAF-V600E, EML4-ALK (E13;A20, E6;A20, E20;A20), KIF5B-RET (K15;R12, K16;R12, K16;R12, K22;R12), CD74-ROS1 (C6;R34), EZR-ROS1 (E10;R34).

13. The method of claim 1, further comprising analyzing the exosome.

14. The method of claim 1, further comprising isolating the exosome from the medium.

15. The method of claim 14, further comprising using the exosome as a reference, a quality control, or a proficiency panel.

16. The method of claim 1, further comprising isolating the RNA from the exosome.

17. The method of claim 16, further comprising detecting the size of the RNA.

18. The method of claim 16, further comprising using the RNA isolated from the exosome as a reference, a quality control, or a proficiency panel.

19. The method of claim 1, further comprising detecting a surface protein on the exosome.

20. The method of claim 19, wherein the surface protein is CD63.

21. The method of claim 1, further comprising detecting the mutation in the RNA.

22. The method of claim 21, wherein the mutation is detected using immuno-histo-chemistry (IHC), fluorescence in situ hybridization (FISH), PCR, Sanger sequencing or next generation sequencing.

23. The method of claim 21, wherein the mutation is detected using RT-PCR, digital PCR, or targeted next generation sequencing.

24. The method of claim 14, further comprising administering the exosome to a subject.

25-30. (canceled)
